(19) United States
(12) Patent Application Publication
(10) Pub. No.: US 2018/0243203 A1
(43) Pub. Date: Aug. 30, 2018

(54) PROCESS FOR TREATING KERATIN FIBRES WITH AN ALKOXYSILANE POLYMER BEARING A NUCLEOPHILIC GROUP AND AN ACTIVATED (THIO)ESTER

(71) Applicant: L'OREAL, Paris (FR)

(72) Inventors: Nawel BAGHDADLI, Massy (FR); Gwenaëlle JEGOU, Saint Michel sur Orge (FR)

(21) Appl. No.: 15/753,283

(22) PCT Filed: Aug. 17, 2016
(86) PCT No.: PCT/EP2016/069535

§ 371 Date: Feb. 17, 2018

(30) Foreign Application Priority Data
Aug. 17, 2015 (FR) 1557769

(51) Int. Cl. A61K 8/898; A61K 8/49; A61Q 5/12; A45D 34/00

(52) U.S. Cl. CPC A61K 8/898; A61K 8/4913; A61K 2800/884; A45D 34/00; A61K 2800/882; A61Q 5/12

(57) ABSTRACT

The present invention relates to a process for treating keratin fibres, comprising the application to the fibres i) of at least one nucleophilic alkoxysilane polymer, in particular at least one aminoalkoxysilane polymer, and ii) at least one aliphatic-chain activated (thio)ester. The invention also relates to a cosmetic composition comprising ingredients i) and ii) and to a kit comprising ingredients i) and ii) for performing such a process. The process gives the treated damaged fibre a long-lasting hydrophobic surface state, the hydrophobicity effect being persistent after one or more shampoo washes, while at the same time affording a good cosmetic feel.

The present invention relates to a process for treating keratin fibres, comprising the application to the fibres i) of at least one nucleophilic alkoxysilane polymer, in particular at least one aminoalkoxysilane polymer, and ii) at least one aliphatic-chain activated (thio)ester. The invention also relates to a cosmetic composition comprising ingredients i) and ii) and to a kit comprising ingredients i) and ii) for performing such a process.

Hair is generally damaged and embrittled by the action of chemical treatments such as dyeing, bleaching, permanent-waving, relaxing and repeated washing.

Is known that certain hair treatments such as dyeing, bleaching, permanent-waving and relaxing can attack the hair fibre and give rise especially to the loss of some of the constituents thereof that are present in the natural state, in particular fatty acids such as 18-methyleicosanoic acid. The hair is thereby damaged and becomes sensitive.

The damaged fibre has an electrostatic nature, making it difficult to correctly shape the hair, especially during combing or brushing. Furthermore, the damaged fibre has a more hydrophilic nature, making it very sensitive to water: the fibre has a tendency to swell and to frizz on contact with ambient moisture, and under these conditions does not ensure good hold of the hairstyle.

It is known that the cosmetic qualities of the hair may be improved by applying various compositions based on active agents or polymers for imparting various properties thereto, such as sheen, ease of disentangling, body, suppleness, liveliness or softness. To obtain good efficacy, these active agents should, of course, have a certain affinity for keratin fibres.

WO 2008/156 327 discloses a composition comprising lipids bearing functional groups, which, after application to the hair or the skin, are covalently bonded to the surface of the keratin materials. The functional groups are, for example, hydroxysuccinimidyl ester groups. The lipids, for their part, are C₈-C₂₈ fatty acids. However, the hydrophobicity effect obtained at the surface of the hair fades out in the course of successive shampoo washes and does not show satisfactory persistence on shampooing.

Moreover, the application of treating polymers to the hair may be harmful to the maintenance of a good feel, especially over the course of successive applications: the treated hair is charged, feels coarse, is not smooth, is grating and is sparingly cosmetic.

WO 2008/1563 and KR 2012-0036877 also disclose processes for treating the hair by applying an activated ester.

Nevertheless, the cosmetic properties of hair treated with these processes are not always satisfactory, especially in terms of hydrophobicity, which may have the effect of increasing the frizziness and reducing the ease of styling and/or the control of the hair volume. These treatments therefore do not make it possible to obtain optimum hydrophobicity and persistence properties. There is thus a need for a hair treatment process that makes it possible to obtain improved hydrophobicity and persistence properties.

Moreover, it is known practice to use aminoalkoxysilanes in hair treatment, as disclosed in U.S. Pat. No. 4,344,763 and US 2009/293899. JP 2012/240 983 describes N-succinimide esters bonded to a silane via an amide function, used for immobilizing proteins as a biosensor substrate.

The object of the present invention is to propose a cosmetic process for treating keratin fibres, especially the hair, making it possible to restore to a damaged fibre the surface physicochemical properties of a natural fibre, and to do so in a long-lasting manner.

A subject of the invention is thus a process for treating keratin materials, comprising:
i) application to the keratin fibres of a cosmetic composition (A) comprising at least one nucleophilic alkoxysilane polymer or oligomer, in particular at least one aminoalkoxysilane polymer; and
ii) application of a cosmetic composition (B) comprising at least one activated (thio)ester compound;
   it being understood that compositions (A) and (B) may be applied to the keratin fibres together or separately, preferably separately.

Another subject of the invention is a cosmetic composition (C) comprising i) at least one nucleophilic alkoxysilane polymer or oligomer, in particular at least one aminoalkoxysilane polymer, and ii) at least one activated (thio)ester compound.

The treatment process is in particular a process for caring for keratin fibres, in particular human keratin fibres such as the hair.

A subject of the invention is also a multi-compartment device or kit, comprising, in a first compartment:
i) at least one nucleophilic alkoxysilane polymer or oligomer, in particular at least one aminoalkoxysilane polymer,
   and, in a second compartment:
ii) at least one activated (thio)ester compound,
   ingredients i) and ii) each being packaged in a separate packaging assembly.

Ingredients i) and ii) of the kit may be in cosmetic compositions (A) and (B) as defined previously. The form of the compositions is known and adapted to be stored (cans, tube, spray can or aerosol can especially).

Such a kit allows the process for treating keratin materials according to the invention to be performed.

The process and the composition according to the invention allow the damaged fibre to regain a hydrophobic surface state close to that of natural hair, and to do so in a long-lasting manner, the hydrophobicity effect being persistent after one or more shampoo washes performed on the treated keratin fibres. The general appearance of the hair is improved, the treated hair is less electrostatic, has less body and less frizziness on contact with ambient moisture, thus contributing to good shaping of the hair, in particular of fine hair. The treated hair also has a good, soft, non-coarse cosmetic feel.

In particular, as shown by the examples, the process and the composition according to the invention make it possible to obtain improved hydrophobicity and persistence properties, while at the same time having a good feel. Application solely of the activated (thio)ester to the hair does not make it possible to achieve the optimum hydrophobicity property, and application solely of the nucleophilic alkoxysilane polymer gives a charged, coarse and thus sparingly cosmetic feel.

For the purposes of the present invention and unless otherwise indicated:
The term “oligomer” means a compound comprising from 2 to 4 repeating monomer units;
The term “polymer” means a compound comprising at least 5 monomer units and preferably between 5 and 50 000 repeating monomer units;
the “aryl” or “heteroaryl” radicals or the aryl or heteroaryl part of a radical may be substituted with at least one substituent borne by a carbon atom, chosen from:
   a C₁-C₈ alkyl radical, optionally substituted with one or more radicals chosen from hydroxyl, C₁-C₂ alkoxy, (poly)hydroxy(C₂-C₄)alkoxy, acylamino, amino substituted with two alkyl radicals;
   a halogen atom;
   a hydroxyl group;
   a C₁-C₂ alkoxy radical;
   a (poly)hydroxy(C₂-C₄)alkoxy radical;
   an amino radical;
   a 5- or 6-membered heterocycloalkyl radical;
   a 5- or 6-membered heteroaryl radical, optionally substituted with a (C₁-C₄)alkyl radical, preferentially methyl;
   an amino radical substituted with one or two identical or different alkyl radicals;
   an acylamino radical (—NR—C(O)—R′) in which the radical R is a hydrogen atom or a C₁-C₄ alkyl radical optionally bearing at least one hydroxyl group and the radical R′ is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)₂—N(R)—) in which the radical R represents a hydrogen atom or a C₁-C₄ alkyl radical optionally bearing at least one hydroxyl group and the radical R′ represents a C₁-C₄ alkyl radical or a phenyl radical; an aminosulfonyl radical ((R)₂N—S(O)₂—) in which the radicals R, which may be identical or different, represent a hydrogen atom or a C₁-C₄ alkyl radical optionally bearing at least one hydroxyl group;
   a carboxyl radical in the acid or salified form (preferably salified with an alkali metal or a substituted or unsubstituted ammonium);
   a cyano group;
   a nitro or nitroso group;
   a polyhaloalkyl group, preferentially the trifluoromethyl group;
the cyclic or heterocyclic part of a non-aromatic radical may be substituted with at least one halogen atom or a substituent chosen from the following groups:
   hydroxyl;
   C₁-C₄ alkoxy, C₂-C₄ (poly)hydroxyalkoxy;
   C₁-C₄ alkyl;
   alkylcarbonylamino (R—C(O)—N(R′)—) in which the radical R′ is a hydrogen atom or a C₁-C₄ alkyl radical optionally bearing at least one hydroxyl group, and the radical R is a C₁-C₂ alkyl radical or an amino radical optionally substituted with one or two C₁-C₄ alkyl groups, which may be identical or different, optionally bearing at least one hydroxyl group, said alkyl radicals possibly forming, with the nitrogen atom to which they are attached a saturated or unsaturated, optionally substituted 5- to 7-membered heterocycle optionally comprising at least one other nitrogen or non-nitrogen heteroatom;
   alkylcarbonyloxy (R—C(O)—O—) in which the radical R is a C₁-C₄ alkyl radical or an amino group optionally substituted with one or two identical or different C₁-C₄ alkyl groups optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
   alkoxycarbonyl (R-G-C(O)—) in which the radical R is a C₁-C₄ alkoxy radical, G is an oxygen atom or an amino group optionally substituted with a C₁-C₄ alkyl group optionally bearing at least one hydroxyl group, said alkyl radical possibly forming with the nitrogen atom to which they are attached a saturated or unsaturated, optionally substituted 5- to 7-membered heterocycle, optionally comprising at least one other nitrogen or non-nitrogen heteroatom;
a cycloalkyl, cycloalkenyl, heterocyclic or heterocycloalkyl radical, or a non-aromatic part of an aryl or heteroaryl radical, may also be substituted with one or more oxo groups;
a hydrocarbon-based chain is unsaturated when it comprises one or more double bonds and/or one or more triple bonds;
an aryl radical represents a monocyclic or fused or non-fused polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;
a heteroaryl radical represents an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;
a cycloalkyl radical is a monocyclic or fused or non-fused, bridged or non-bridged polycyclic hydrocarbon-based radical containing from 5 to 22 carbon atoms, such as cyclohexyl;
a cycloalkenyl radical is a monocyclic or fused or non-fused, bridged or non-bridged polycyclic non-aromatic unsaturated hydrocarbon-based radical containing from 5 to 22 carbon atoms, such as cyclohexenyl;
a heterocycloalkyl radical is a heterocyclic radical comprising at least one saturated ring;
a heterocyclic radical is a 5- to 22-membered monocyclic or fused or non-fused polycyclic radical which may contain one or two unsaturations but is non-aromatic, comprising from 1 to 6 heteroatoms chosen from nitrogen, oxygen, sulfur and selenium atoms;
an alkyl radical is a linear or branched C₁-C₂₀ and preferably C₁-C₈ hydrocarbon-based radical;
the term optionally substituted attributed to the alkyl radical or a hydrocarbon-based aliphatic chain means that said radical or said chain may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom; v) or an aryl group such as phenyl;
an alkoxy radical is an alkyl-oxy radical for which the alkyl radical is a linear or branched C₁-C₁₆ and preferentially C₁-C₈ hydrocarbon-based radical;
when the alkoxy group is optionally substituted, this implies that the alkyl group is optionally substituted as defined hereinabove;
the term organic or mineral acid salt more particularly means the salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
moreover, the addition salts that may be used in the context of the invention are especially chosen from addition salts with a cosmetically acceptable base such as basifying agents as defined below, for instance alkali metal hydroxides, such as sodium hydroxide or potassium hydroxide, aqueous ammonia, amines or alkanolamines;
the expression at least one is equivalent to one or more; and
the expression inclusive for a range of concentrations means that the limits of the range are included in the defined range.
i) Nucleophilic alkoxysilane polymer or oligomer

The process, the composition and the kit according to the invention use i) at least one nucleophilic alkoxysilane polymer or oligomer.

The term nucleophilic alkoxysilane polymer or oligomer means any alkoxysilane polymer or oligomer derived from the polymerization of a silane monomer which comprises a) at least one (C₁-C₁₀)alkoxy group and b) at least one “nucleophilic” group, i.e. an electron-donating group such as amino, hydroxyl or thiol, said electron donor comprising a hydrogen atom that can be removed in basic medium.

More particularly, the nucleophilic group(s) are primary or secondary amino groups —N(H)R_{b} with R_{b} representing a hydrogen atom or an optionally substituted (C₁-C₆)alkyl group, a (C₆-C₁₀)cycloalkyl group, a 5- to 10-membered heterocycloalkyl group or a (hetero)aryl group.

According to a particular embodiment of the invention, the nucleophilic alkoxysilane polymer or oligomer is derived from a monomer of formula (I) below, and also the acid salts thereof, and the solvates thereof such as hydrates:

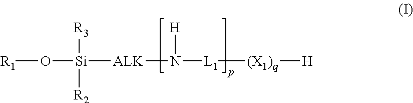

in which formula (I):
p and q, which may be identical or different, are equal to 0 or 1, with the sum p+q being greater than or equal to 1, preferably p+q is equal to 1 or 2, in particular p is 0 and q is 1;
X₁ represents an oxygen or sulfur atom or an amino group —N(Rₐ)— with Rₐ representing a hydrogen atom or a group chosen from (C₁-C₈)alkyl such as methyl, (C₅-C₇)cycloalkyl such as cyclohexyl, aryl such as phenyl, and aryl(C₁-C₄)alkyl such as benzyl, in particular an amino group and preferably —N(H)—;
ALK represents a linear or branched C₁-C₁₀, in particular linear, saturated divalent hydrocarbon-based chain, more particularly —(CH₂)ₓ— with x representing an integer between 1 and 6 inclusive, preferably between 1 and 4 such as methylene, ethylene or propylene; more preferentially methylene or propylene, or even propylene;
R₁ represents a hydrogen atom or a (C₁-C₆)alkyl group such as methyl or ethyl;
R₂ and R₃, which may be identical or different, preferably identical, represent a group chosen from hydroxyl, (C₁-C₆)alkyl and (C₁-C₆)alkoxy, in particular C₁-C₄ alkoxy such as ethoxy;
L₁ represents a linear or branched saturated C₁-C₂₀ divalent hydrocarbon-based chain; in particular linear, more particularly —(CH₂)ₓ— with x as defined previously; in particular, L₁ is an ethylene or propylene group.

According to a particular embodiment of the invention, the nucleophilic alkoxysilane polymer(s) or oligomer(s) are derived from monomers of formula (I) as defined previously in which, taken together or separately:
p is 0 and q is 1;
R₁ represents a (C₁-C₄)alkyl group such as ethyl;
R₂ and R₃ are identical and represent a (C₁-C₄)alkoxy group such as ethoxy;
ALK represents a methylene or propylene group, preferably propylene;
X₁ represents an amino group —N(Rₐ)— as defined previously, in particular, Rₐ representing a hydrogen atom or a (C₁-C₄)alkyl group; preferably, Rₐ represents a hydrogen atom.

Advantageously, the nucleophilic alkoxysilane polymer or oligomer has a weight-average molecular weight ranging from 200 to 1 000 000 g/mol, preferably ranging from 300 to 500 000 g/mol.

Preferably, the nucleophilic alkoxysilane polymer or oligomer is water-soluble. The term “water-soluble polymer” means a polymer with a solubility in water, at 25° C., of at least 0.1 g/I.

The nucleophilic alkoxysilane polymer or oligomer may be present in the composition in which said polymer is contained in a content ranging from 0.001% to 20% by weight, relative to the total weight of the composition in which said polymer is contained, preferably ranging from 0.001% to 10% by weight, preferentially ranging from 0.001% to 5% by weight, and more preferentially ranging from 0.5% to 5% by weight.

Preferably, they are oligomers or polymers obtained by polymerization of APTES.
ii) an activated (thio)ester

The process, the composition and the kit according to the invention also use ii) at least one activated (thio)ester compound.

The term “activated (thio)ester compound” means an ester or thioester compound comprising an aliphatic chain of formula R—C(Y)—Y′-A with:
R representing a linear or branched, saturated or unsaturated, preferably saturated, optionally substituted, preferably unsubstituted, hydrocarbon-based aliphatic chain, optionally interrupted with one or more heteroatoms such as oxygen, comprising from 5 to 30 carbon atoms;
Y and Y′, which may be identical or different, represent an oxygen or sulfur atom; in particular, Y′ represents an oxygen atom; and
A representing an activating group of the carbon atom C of the (thio)carbonyl group —C(Y)—, which is able to leave in the presence of a nucleophile, liberate A-Y′⁻ and create a covalent bond between the carbon atom C of the —C(Y)— group and said nucleophile.

In particular, the group A is an optionally substituted heterocyclic group or an optionally substituted heteroaryl group linked to the rest of the molecule via a heteroatom such as nitrogen, or a (hetero)arylamino group.

According to a particular embodiment of the invention, the activated (thio)ester(s) are of formula (II) below, and also the solvates thereof such as hydrates:

R′—C(Y)—OA₁ (II)

in which formula (II):
R′ represents a C₅-C₂₁, preferably C₉-C₁₇ and preferentially C₁₁-C₁₅ alkyl group;
Y is as defined previously; preferably, Y represents an oxygen atom;
A₁ denotes a reactive group chosen from A₁ₐ, A_{1b} and A_{1c}:

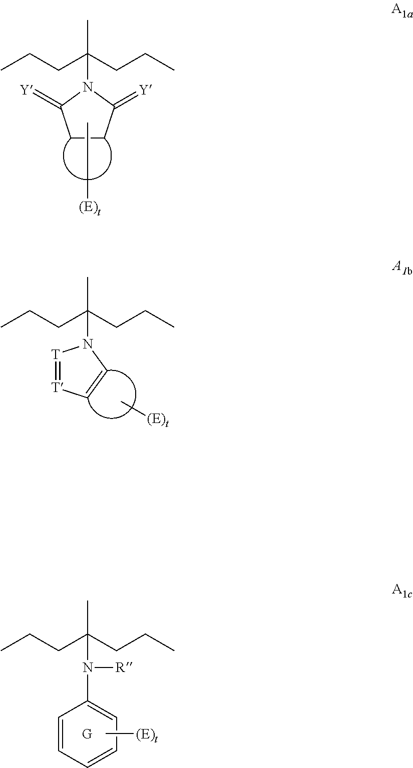

with
   Y′, which may be identical or different, preferably identical, representing an oxygen or sulfur atom, preferably oxygen;

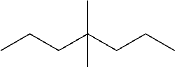

representing the bond which links A₁ₐ, A_{1b} or A_{1c} to the rest of the molecule;
T and T′, which may be identical or different, preferably identical, representing a methylene group C(R^{a}) with R^{a} representing a hydrogen atom or an optionally substituted (C₁-C₆)alkyl, or a nitrogen atom, in particular nitrogen, or alternatively T represents a group C(R^{a}) such as CH and T′ represents a nitrogen atom;
E, which may be identical or different, when t is greater than or equal to 2, representing an atom or electron-withdrawing group such as halogen, nitro, nitroso, cyano, carboxyl, phosphate, polyhaloalkyl, such as trifluoromethyl, sulfoxy, SO₃⁻M⁺ with M⁺ representing a hydrogen atom, or a cationic counterion such as alkali metal, alkaline-earth metal or ammonium, such as sodium or potassium; preferably, E represents a halogen atom such as fluorine or chlorine or a group SO₃⁻M⁺;
t representing an integer between 0 and 5 inclusive;
G representing an aryl group such as phenyl, or a heteroaryl group;

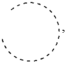

which may be present or absent, represents a (C₅-C₁₀)cycloalkyl, (C₅-C₁₀)cycloalkenyl, 5- to 10-membered heterocycloalkyl, 5- to 10-membered heterocycloalkenyl, aryl such as benzo, or heteroaryl monocycle or fused bicycle; preferably a fused bicyclic (C₅-C₇)cycloalkyl, bicyclic (C₅-C₇)cycloalkenyl or benzo group; and
R″ representing a hydrogen atom or an optionally substituted (C₁-C₆)alkyl group such as benzyl, or a (hetero)aryl group such as phenyl;

According to a particular embodiment of the invention, the polyalkoxylated activated (thio)ester(s) are of formula (II) as defined previously in which A₁ are chosen from A′₁ₐ, A″₁ₐ, A′″₁ₐ, A″″₁ₐ, A′_{1b}, A″_{1b} and A′_{1c}:

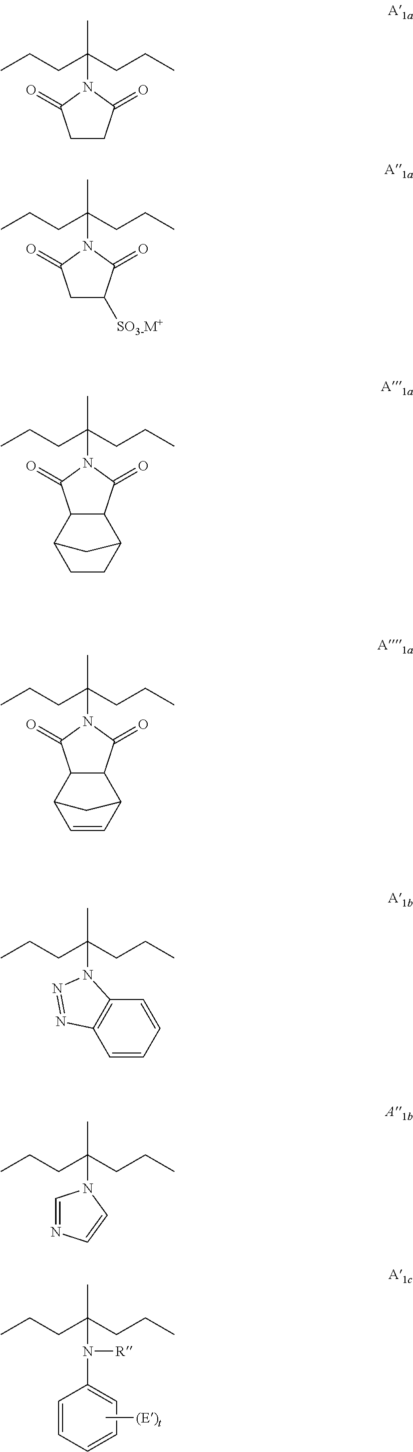

with:
M⁺ representing a cationic counterion as defined previously, in particular representing an alkali metal such as sodium;
E′, which may be identical or different, when t is greater than or equal to 2, representing an electron-withdrawing group such as a halogen atom, chosen in particular from fluorine and chlorine;
t is an integer between 0 and 5 inclusive;
R″ being as defined previously, in particular representing a hydrogen atom or a (C₁-C₆)alkyl group, preferably a hydrogen atom.

Preferably, A₁ represents a group A′₁ₐ,A″₁ₐ, or A″_{1b}, preferably A′₁ₐ.

These activated (thio)ester compounds are known from the literature and to those skilled in the art.

More particularly, A₁ represents a group A′₁ₐ or A″₁ₐ, preferably A′₁ₐ and R′ represents a C₉-C₁₇ and preferentially C₁₁-C₁₅ alkyl group.

Among the reactive groups, the activated (thio)ester compound may be:

the esters of lauric, palmitic, pentanoic, hexanoic, 2-ethylhexanoic, octanoic, nonanoic, decanoic, dodecanoic, hexadecanoic, eicosanoic, hexacosanoic, octadecenoic, undecenoic, eicosatetraenoic or octadecatrienoic acid and of N-hydroxysuccinimide,

The activated (thio)ester compound is preferably chosen from: the ester of lauric acid and of N-hydroxysuccinimide, the ester of palmitic acid and of N-hydroxysuccinimide. Advantageously, the activated (thio)ester compound is the ester of palmitic acid and of N-hydroxysuccinimide. The activated (thio)ester compound may be present in the composition in which said activated (thio)ester compound is contained in a content ranging from 0.1% to 5% by weight, relative to the total weight of the composition in which said activated (thio)ester compound is contained, preferably ranging from 0.5% to 4% by weight, and preferentially ranging from 1% to 4% by weight.

### Composition (C) or Compositions (A) and (B)

Composition (C) or compositions (A) and (B) used according to the invention contain a physiologically acceptable medium, i.e. a medium that is compatible with human keratin materials such as the skin (of the body, face, eye contour area or the scalp), the hair, the eyelashes, the eyebrows, bodily hair, the nails or the lips.

Advantageously, the cosmetic composition (A) according to the invention comprises a physiologically acceptable aqueous medium. It may be constituted, for example, of water or of a mixture of water and of at least one cosmetically acceptable organic solvent. Examples of organic solvents that may be mentioned include C₂-C₄ lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether, diethylene glycol monomethyl ether or monoethyl ether, and short esters such as ethyl acetate or butyl acetate; and mixtures thereof. Preferably, the cosmetic composition comprises from 50% to 99.5% by weight of water relative to the weight of the composition.

Advantageously, the cosmetic composition (B) used according to the invention comprises a physiologically acceptable non-aqueous medium. It may be constituted, for example, by one or more cosmetically acceptable organic solvents, such as those described previously, or alternatively one or more common cosmetic oils.

Advantageously, the cosmetic composition (C) comprises a physiologically acceptable aqueous medium. It may be constituted, for example, by water or by a mixture of water and of at least one cosmetically acceptable organic solvent as described previously, or alternatively one or more common cosmetic oils. Preferably, the cosmetic composition (C) comprises from 40% to 99.5% by weight of water relative to the weight of the composition.

Composition (C) or compositions (A) and (B) used according to the invention may also contain one or more cosmetic additives chosen from nonionic, anionic, cationic and amphoteric surfactants, vitamins and provitamins, including panthenol, sunscreens, fillers, colorants, nacreous agents, opacifiers, sequestrants, film-forming polymers, plasticizers, thickeners, aliphatic or silicone oils, antioxidants, antifoams, moisturizers, emollients, penetrants, fragrances and preserving agents.

Composition (C) or compositions (A) and (B) used according to the invention may be in any galenical form conventionally used for application to the hair and especially in the form of aqueous solutions, aqueous-alcoholic solutions, oil-in-water (O/W), water-in-oil (W/O) or multiple (triple: W/O/W or O/W/O) emulsions, aqueous gels or aqueous-alcoholic gels. These compositions are prepared according to the usual methods. Preferably, the composition is in the form of an aqueous or aqueous-alcoholic solution or gel.

### The Process for Treating Keratin Fibres

The process according to the invention is performed on keratin fibres, especially human keratin fibres such as the hair. Said fibres may be wet or dry. Preferentially, the process is performed on dry keratin fibres, especially hair.

According to a particular embodiment of the invention, ingredients i) and ii) as defined previously are together in the same cosmetic composition (C) that is then applied to the keratin fibres. In this case, composition (C) may originate from the extemporaneous mixing of compositions (A) and (B). According to one embodiment, compositions (A) and (B) are applied together to the keratin fibres, compositions (A) and (B) may be mixed together before application to give a composition (C), said composition (C) then being applied to the keratin fibres.

According to a particularly advantageous embodiment, compositions (A) and (B) are applied to the keratin fibres separately.

According to one embodiment of the process of the invention, composition (B) is applied first to the keratin fibres, and composition (A) is subsequently applied to the keratin fibres.

According to a preferred embodiment of the process of the invention, composition (A) is applied to the keratin fibres and composition (B) is subsequently applied to the keratin fibres.

Preferably, when the process for treating keratin fibres of the invention is performed in several steps, between the step of applying composition (A) and composition (B), or between the step of applying composition (B) and composition (A), no intermediate rinsing with water is performed.

According to a particularly preferred embodiment of the process of the invention, composition (A) is applied to the keratin fibres, then keratin fibres are dried, and composition (B) is subsequently applied to the keratin fibres.

According to another embodiment of the process of the invention, composition (B) is applied first to the keratin fibres, then keratin fibres are dried and composition (A) is subsequently applied to the keratin fibres.

The drying step is preferably complete. The latter can be performed at room temperature or by heat treatment, preferably at room temperature. The heat treatment is conducted at a temperature between 30 and 250° C. until completely dry. In practice, this can be conducted using a hair helmet, hair dryer, a curl or flat iron, an infrared rays dispenser or other heating devices and under plastic film, preferably with a domestic hairdryer in particular at a temperature between 50° C. and 100° C.

After application to the keratin fibres of the cosmetic composition (A), (B) or (C), said applied composition may be left to stand on the fibres for a time ranging from 1 to 60 minutes, preferably ranging from 2 to 50 minutes, preferentially ranging from 5 to 30 minutes. The leave-on time may take place at a temperature ranging from 15° C. to 45° C., preferably at room temperature (25° C.).

The cosmetic composition(s) described previously are advantageously applied to the keratin fibres in an amount ranging from 0.1 to 10 grams and preferably from 0.2 to 5 grams of composition per gram of keratin fibres.

After application of the cosmetic compositions to the keratin fibres, the latter may be drained dry to remove the excess composition or washed with water.

After the treatment, the keratin fibres may be optionally rinsed with water or washed with a shampoo. The keratin fibres are then optionally dried with a hairdryer or a hood or in the open air.

The treatment process according to the invention is preferably performed on keratin fibres, especially hair, which are sensitized, such as artificially dyed fibres (keratin fibres dyed following a direct dyeing process or via an oxidation dyeing process), bleached, relaxed or permanent-waved fibres.

The treatment process according to the invention may be performed before, during and/or after an additional process of cosmetic treatment of the keratin fibres, such as a process for temporarily shaping (shaping with curlers, a crimping iron or a straightening iron) or a process for durably shaping (permanent-waving or relaxing) the keratin fibres.

The treatment process may be performed as a pre-treatment to a dyeing or relaxing process and/or a permanent-waving process so as to cosmetically protect the keratin fibres against these treatments. In other words, this process is performed to preserve the cosmetic properties of the keratin fibres before a cosmetic treatment process as described previously.

Preferentially, the treatment process is performed as a post-treatment to a bleaching, artificial dyeing or relaxing process and/or a permanent-waving process so as to repair said fibres.

The process according to the invention is preferably performed on the hair.

The examples that follow are given as illustrations of the present invention.

The amounts indicated in the examples are expressed as weight percentages.

### EXAMPLES

### Process for Treating Keratin Fibres

### Process 1 in 2 Steps:

1) Treatment of the keratin fibre lock with a solution A of nucleophilic alkoxysilane polymer:

The lock was moistened, and 1.5 g of an aqueous solution of (3-aminopropyl)triethoxysilane (APTES) at 10% by weight/g of hair were then deposited (the aqueous composition with APTES was applied by pipette and then by brush onto the lock, in a relatively wide plate, so as to spread out the lock as much as possible).

The lock was then placed in an oven at 40° C. for 30 minutes covered with aluminium foil to prevent evaporation, and the lock was then turned over after 15 minutes without intermediate rinsing.
2) Subsequent treatment of the keratin fibre lock with a solution B of activated (thio)ester compound

A solution of “Palmitic NHS”, ester of palmitic acid and of N-hydroxysuccinimide, at 3% by weight in butyl acetate/ethanol medium (50/50 v/v) was prepared, and 1.5 g of this solution/g of hair were then deposited. The application method is identical to that of the preceding step.

The lock was then placed in an oven at 40° C. for 60 minutes covered with aluminium foil to prevent evaporation, and the lock was then turned over after 30 minutes. The lock was divided into two, one part was rinsed and the other was not.

### Process 2 in 2 Steps:

1) Treatment of the keratin fibre lock with a solution B of activated (thio)ester compound

The lock was moistened, and 1.5 g of a solution of “Palmitic NHS” at 3% by weight in butyl acetate/ethanol medium (50/50 v/v)/g of hair were then applied. The application method and the conditions are identical to those of step 1) of the preceding process 1.
2) Subsequent treatment of the keratin fibre lock with a solution A of nucleophilic alkoxysilane polymer:

1.5 g of an aqueous solution of APTES at 10% by weight/g of hair were applied under the same experimental conditions as in step 2) of the preceding process 1.

### Process 3 in 1 Step:

### Preparation of Composition C:

A solution of APTES at 10% by weight in a butyl acetate/ethanol mixture (50/50 v/v) is prepared. Another solution comprising Palmitic NHS at 3% by weight in a butyl acetate/ethanol mixture (50/50 v/v) is prepared. The two solutions are then mixed together in a 50/50 v/v amount, to give composition C.

3 g of composition C are then applied to 1 g of a lock of hair. The application method and the experimental conditions are identical to those of step 2) of the preceding processes 1 and 2.

### Process 4 in 3 Steps:

1) Treatment of the keratin fibre lock with a solution A of nucleophilic alkoxysilane polymer:

The lock was moistened, and 1.5 g of an aqueous solution of (3-aminopropyl)triethoxysilane (APTES) at 10% by weight/g of hair were then deposited. The application method and the conditions are identical to those of step 1) of the preceding process 1.
2) Subsequent drying of the keratin fibre lock at room temperature (25° C.)
3) Subsequent treatment of the keratin fibre lock with a solution B of activated (thio)ester compound

A solution of “Palmitic NHS”, ester of palmitic acid and of N-hydroxysuccinimide, at 3% by weight in butyl acetate/ethanol medium (50/50 v/v) was prepared, and 1.5 g of this solution/g of hair were then deposited. The application method is identical to that of the preceding step.

The lock was then placed in an oven at 40° C. for 60 minutes covered with aluminium foil to prevent evaporation, and the lock was then turned over after 30 minutes. The lock was then rinsed once with a shampoo.

### Evaluations

The various conditions that were used are as follows:

Four controls/placebos:
H₂O control corresponding to the untreated hair
Mixture of butyl acetate/ethanol (50/50 v/v) corresponding to the palmitic-NHS solvent for examination of the reactivity of the solvent alone
APTES (10% by weight in water H₂O) for examination of the reactivity of “APTES” alone
palmitic-NHS (noted -NHS) (3% by weight in a butyl acetate/ethanol mixture (50/50 v/v)) for examination of the reactivity of NHS alone

Four treatment conditions were implemented:
Condition in 2 stages: APTES (10%) followed by palmitic-NHS (3%) corresponding to process 1
Condition in 2 stages: palmitic-NHS (3%) then APTES (10%) corresponding to process 2
Condition in 1 stage: aqueous solution of APTES (10%) and palmitic-NHS (3%) corresponding to process 3
Condition in 3 stage: aqueous solution of APTES (10%) followed by a drying step at room temperature and followed by palmitic-NHS (3%) corresponding to process 4

The locks were treated with the compositions prepared according to the protocol and the process described above.

| | | Invention/ |
|---|---|---|
| Locks | Treatment | Comparative |
| Lock 1 | H₂O - Control | Comparative |
| Lock 2 | Butyl acetate/ethanol (50/50 v/v) | Comparative |
| Lock 3 | Palmitic NHS (3% by mass in a 50/50 v/v butyl | Comparative |
| | acetate/ethanol mixture) | |
| Lock 4 | “APTES” water into which 10% by weight of | Comparative |
| | APTES was introduced | |
| Lock 5 | Process 1 - APTES followed by palmitic NHS | Invention |
| Lock 6 | Process 2 - palmitic NHS followed by APTES | Invention |
| Lock 7 | Process 3 - APTES and palmitic NHS together | Invention |
| Lock 8 | Process 4 - APTES followed by a drying step at | Invention |
| | room temperature and followed by palmitic-NHS | |

### Wetting Measurements: Surface Hydrophobicity/Hydrophilicity

For each lock of treated hair, the wettability of the hair was measured (measurements described especially in the book The Science of Hair Care by C. Bouillon and J. Wilkinson—2^{nd} edition 2005—Chapter 12: Evaluation of product efficacy—page 407).

The measurement of the wettability of a hair consists in immersing a piece of hair in a crystallizing dish of ultra-pure water and in measuring the force generated by the displacement of the hair fibre during its immersion (wetting force) and during its withdrawal from the water. This force varies as a function of the affinity of the hair for the liquid and makes it possible to assess the surface state of the fibre.

A K14 tensiometer from the company Krüss was used under the following operating conditions:
Approach speed: 4 mm/minute.
Measuring speed: 2 mm/minute.
Balance sensitivity: 10 μg
Waiting time after immersion: 2 seconds
Immersion depth: 2 mm

The hairs were raised on a holder still oriented in the same direction, i.e. end downwards, root upwards.

For each treatment, 20 samples were measured under the same conditions.

The hairs were left to regulate overnight in a glove box at 25° C. and 45% hygrometry before measuring them.

### Data Processing

The wetting force (F in newtons) was determined according to the following formula:

F=L×σ×cos θ
L being the perimeter of the hair (in metres)
θ: contact angle
Σ: surface tension of pure water=72.75 mN/m at atmospheric pressure

The wetting force expressed in mN was transformed into μg, and then normalized relative to the mean perimeter of the fibres.

From the 20 measurements for each lock, the mean value and its confidence interval were calculated. The lower the wetting force, the more hydrophobic the treated hair (less frizziness, better manageability, easier to style, volume control).

### Friction Measurements: Slipperiness

The slipperiness of a treatment according to the invention is measured using a friction banc compared with a H₂O control (corresponding to the untreated hair) and an APTES control (10% by weight in water H₂O).

Conditions for measuring 2 hair:
Base weight: P=11 g
Measurement length: 3 mm
Friction Speed: 1 mm/min
Frequency: 20 Hz
RH: 50%

Total number of measurements 16 hairs/condition and treatment

The frictional force between the blade and the moving hairs is then measured. More friction force between two surfaces, the lower the slipperiness of the treatment is important. The friction force is expressed in Newtons (N), and friction coefficient μ is calculated according to the following formula: Amonton's law:

F_{H}=μ×P

### Results:

It was seen that process 1 for treating keratin fibres made it possible to obtain smooth-feeling hair combining a body effect and coating. In addition, it was observed that this sensation persists even after several shampoo washes.

| | No shampoo | Rinsing + 1 | Rinsing + 5 |
|---|---|---|---|
| | washes | shampoo wash | shampoo washes |
| Locks | F (μg) | F (μg) | F (μg) |
| 1 (control) | 7.53 ± 0.99 | 9.84 ± 0.52 | 6.86 ± 0.52 |
| 2 | 8.53 ± 0.63 | 8.92 ± 0.34 | 7.50 ± 0.45 |
| (comparative) | | | |
| 3 | 8.38 ± 0.63 | 6.94 ± 0.44 | 4.15 ± 0.36 |
| (comparative) | | | |
| 4 | −2.11 ± 0.98 | −0.61 ± 0.83 | −5.35 ± 0.98 |
| (comparative) | | | |
| 5 (invention) | −11.64 ± 0.73 | −6.39 ± 0.59 | −6.46 ± 0.21 |
| 6 (invention) | −9.84 ± 0.95 | −7.82 ± 0.47 | −2.18 ± 0.32 |
| 7 (invention) | −4.17 ± 1.16 | −9.29 ± 0.37 | −4.96 ± 0.31 |

It was seen that the process according to the invention made it possible significantly to afford long-lasting hydrophobic nature to the surface of the keratin fibres, and also a good feel.

It was seen that the process 4 for treating keratinous fibres, according to the invention, provides improved slipperiness, and therefore gives the hair a smooth-feeling and makes it easier to detangle the hair.

| | Frictional | |
|---|---|---|
| | force Direction: | Frictional force |
| Locks | from root to tip | Direction: from tip to root |
| Lock 1 - comparative | 0.208 | 0.227 |
| Lock 4 - comparative | 0.194 | 0.200 |
| Lock 8 - invention | 0.173 | 0.196 |

The slipperiness of the treatment, according to the invention, is very attractive, since it has a friction force less than the controls, regardless of the measuring direction.

1-17. (canceled)

18. A method for treating keratin fibers, comprising:
i) applying to the keratin fibers a cosmetic composition (A) comprising at least one nucleophilic alkoxysilane polymer or oligomer; and
ii) applying to the keratin fibers a cosmetic composition (B) comprising at least one activated (thio)ester compound according to formula R—C(Y)—Y′-A, wherein:
R is chosen from a linear or branched, saturated or unsaturated, optionally substituted, hydrocarbon-based aliphatic chain, optionally interrupted with at least one heteroatom, comprising from 5 to 30 carbon atoms;
Y and Y′, which may be identical or different, are chosen from an oxygen or sulfur atom; and
A is an activating group of the carbon atom C of the (thio)carbonyl group —C(Y)—, which is able to leave in the presence of a nucleophile, liberate A-Y′⁻, and create a covalent bond between the carbon atom C of the —C(Y)— group and said nucleophile;
wherein compositions (A) and (B) may be applied to the keratin fibers together or separately.

19. The method according to claim 18, wherein the at least one nucleophilic alkoxysilane polymer or oligomer comprises at least one nucleophilic group chosen from primary or secondary amino groups —N(H)R_{b}, wherein R_{b} is chosen from a hydrogen atom, an optionally substituted (C₁-C₆)alkyl group, a (C₅-C₁₀)cycloalkyl group, a 5- to 10-membered heterocycloalkyl group, or a (hetero)aryl group.

20. The method according to claim 18, wherein the at least one nucleophilic alkoxysilane polymer or oligomer is derived from a monomer of formula (I) below, the acid salts thereof, or solvates thereof: 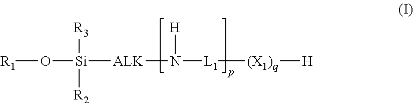
wherein:
p and q, which may be identical or different, are equal to 0 or 1, with the sum p+q being greater than or equal to 1;
X₁ is chosen from an oxygen or sulfur atom or an amino group —N(Rₐ)— wherein Rₐ is chosen from a hydrogen atom, a (C₁-C₈)alkyl group, a (C₅-C₇)cycloalkyl group, an aryl group, or an aryl(C₁-C₄)alkyl group;
ALK is chosen from a linear or branched C₁-C₁₀ group;
R₁ is chosen from a hydrogen atom or a (C₁-C₆)alkyl group;
R₂ and R₃, which may be identical or different, are chosen from hydroxyl, a (C₁-C₆)alkyl group, or a (C₁-C₆)alkoxy group; and
L₁ is chosen from a linear or branched, saturated C₁-C₂₀ divalent hydrocarbon-based chain.

21. The method according to claim 20, wherein the at least one nucleophilic alkoxysilane polymer or oligomer is derived from a monomer of formula (I), the acid salts thereof, or solvates thereof, wherein:
p is 0 and q is 1;
R₁ is chosen from a (C₁-C₄)alkyl group;
R₂ and R₃ are identical and chosen from a (C₁-C₄)alkoxy group;
ALK is chosen from a methylene or propylene group; and
X₁ is an amino group —N(Rₐ)— wherein Rₐ is chosen from a hydrogen atom, a (C₁-C₈)alkyl group, a (C₅-C₇)cycloalkyl group, an aryl group, or an aryl(C₁-C₄)alkyl group.

22. The method according to claim 18, wherein group A of the at least one activated (thio)ester compound is chosen from:
an optionally substituted heterocyclic group linked to the rest of the molecule via a heteroatom such as nitrogen;
an optionally substituted heteroaryl group linked to the rest of the molecule via a heteroatom such as nitrogen; or
a (hetero)arylamino group.

23. The method according to claim 18, wherein the at least one activated (thio)ester compound is chosen from compounds according to formula (II) below, or solvates thereof:
R′—C(Y)—OA₁ (II)
wherein:
R′ is chosen from a C₅-C₂₁ group;
Y is chosen from an oxygen or sulfur atom;
A₁ is a reactive group chosen from A₁ₐ, A_{1b}, or A_{1c}, below: 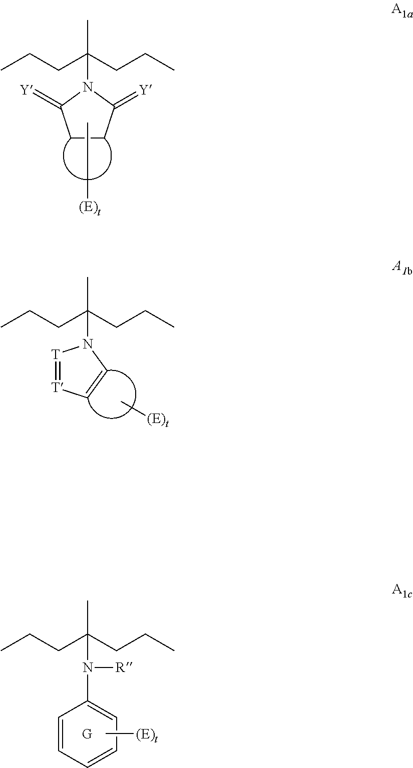
wherein:
Y′, which may be identical or different, is chosen from an oxygen or sulfur atom; 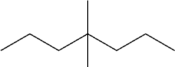
represents the bond which links A₁ₐ, A_{1b}, or A_{1c} to the rest of the molecule;
T and T′, which may be identical or different, are chosen from a methylene group C(R^{a}) wherein R^{a} is chosen from a hydrogen atom, an optionally substituted (C₁-C₆)alkyl group, or a nitrogen atom, or alternatively T is chosen from a C(R^{a}) group and T′ is a nitrogen atom;
E, which may be identical or different, when t is greater than or equal to 2, is chosen from an atom or electron-withdrawing group, chosen from halogen, nitro, nitroso, cyano, carboxyl, phosphate, polyhaloalkyl, sulfoxy, SO₃⁻M⁺ with M⁺ representing a hydrogen atom, or a cationic counterion;
t is an integer between 0 and 5 inclusive;
G is chosen from an aryl group or a heteroaryl group; 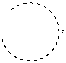
which may be present or absent, is chosen from (C₅-C₁₀)cycloalkyl, (C₅-C₁₀)cycloalkenyl, 5- to 10-membered heterocycloalkyl, 5- to 10-membered heterocycloalkenyl, aryl, a heteroaryl monocycle, or fused bicycle; and
R″ is chosen from a hydrogen atom or an optionally substituted (C₁-C₆)alkyl group.

24. The method according to claim 23, wherein the at least one activated (thio)ester compound is chosen from compounds according to formula (II), or solvates thereof, wherein A₁ is chosen from A′₁ₐ, A″₁ₐ, A′″₁ₐ, A″″₁ₐ, A′_{1b}, A″_{1b}, or A′_{1c}: 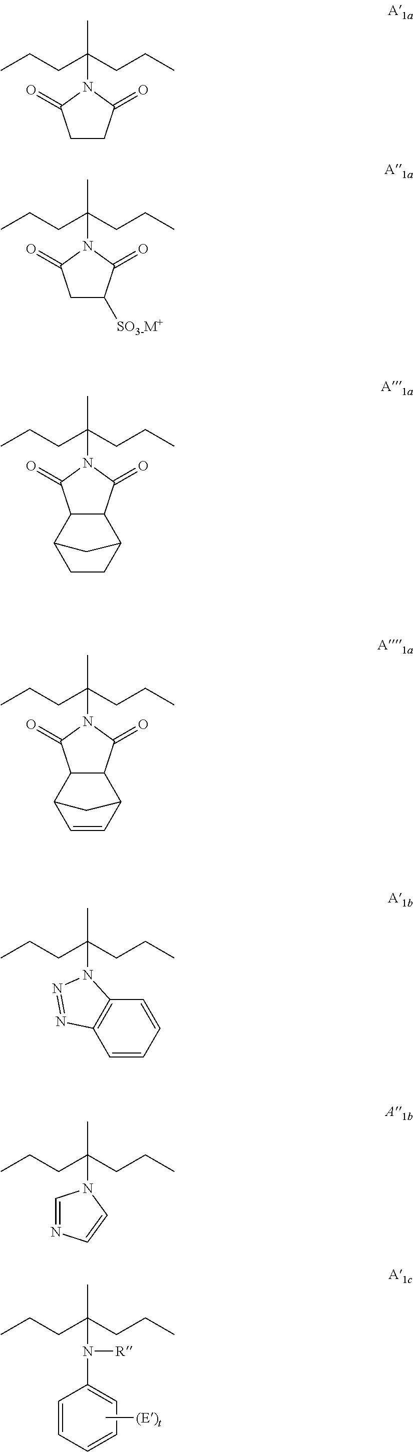
wherein:
M⁺ is chosen from a cationic counterion;
E⁺, which may be identical or different, when t is greater than or equal to 2, is chosen from an electron-withdrawing group;
t is an integer between 0 and 5 inclusive; and
R″ is chosen from a hydrogen atom or an optionally substituted (C₁-C₆)alkyl group.

25. The method according to claim 23, wherein the at least one activated (thio)ester compound is chosen from compounds according to formula (II) wherein A₁ is chosen from A′₁ₐ or A″₁ₐ.

26. The method according to claim 18, wherein the at least one activated (thio)ester compound is chosen from esters of lauric, palmitic, pentanoic, hexanoic, 2-ethylhexanoic, octanoic, nonanoic, decanoic, dodecanoic, hexadecanoic, eicosanoic, hexacosanoic, octadecenoic, undecenoic, eicosatetraenoic, or octadecatrienoic acid, or esters of N-hydroxysuccinimide.

27. The method according to claim 18, wherein the at least one activated (thio)ester compound is present in an amount ranging from about 0.1% to about 5% by weight, relative to the total weight of the composition.

28. The method according to claim 18, comprising:
mixing compositions (A) and (B) prior to application to form composition (C);
and applying composition (C) to the keratin fibers.

29. The method according to claim 18, comprising:
first, applying composition (B) to the keratin fibers; and
second, applying composition (A) to the keratin fibers,
wherein the keratin fibers are not rinsed between the application of composition (B) and composition (A).

30. The method according to claim 18, comprising:
first, applying composition (A) to the keratin fibers; and
second, applying composition (B) to the keratin fibers,
wherein the keratin fibers are not rinsed between the application of composition (A) and composition (B).

31. The method according to claim 18, wherein composition (A) and composition (B) are applied to the keratin fibers separately, further comprising drying the keratin fibers between the applications of the compositions.

32. The method according to claim 18, further comprising:
leaving the composition(s) on the keratin fibers for a leave-on time ranging from about 1 minute to about 60 minutes;
optionally rinsing the keratin fibers; and
optionally drying the keratin fibers.

33. The method according to claim 18, wherein the method is performed on the keratin fibers before, during, and/or after an additional, but different, method of cosmetic treatment on the keratin fibers.

34. A cosmetic composition comprising:
i) at least one nucleophilic alkoxysilane polymer or oligomer; and
ii) at least one activated (thio)ester compound according to formula R—C(Y)—Y′-A, wherein:
R is chosen from a linear or branched, saturated or unsaturated, optionally substituted, hydrocarbon-based aliphatic chain, optionally interrupted with at least one heteroatom, comprising from 5 to 30 carbon atoms;
Y and Y′, which may be identical or different, are chosen from an oxygen or sulfur atom; and
A is an activating group of the carbon atom C of the (thio)carbonyl group —C(Y)—, which is able to leave in the presence of a nucleophile, liberate A-Y′⁻, and create a covalent bond between the carbon atom C of the —C(Y)— group and said nucleophile.

35. A device, comprising:
in a first compartment, i) at least one nucleophilic alkoxysilane polymer or oligomer; and
in a second compartment, ii) at least one activated (thio)ester compound according to formula R—C(Y)—Y′-A, wherein:
R is chosen from a linear or branched, saturated or unsaturated, optionally substituted, hydrocarbon-based aliphatic chain, optionally interrupted with at least one heteroatom, comprising from 5 to 30 carbon atoms;
Y and Y′, which may be identical or different, are chosen from an oxygen or sulfur atom; and
A is an activating group of the carbon atom C of the (thio)carbonyl group —C(Y)—, which is able to leave in the presence of a nucleophile, liberate A-Y′⁻, and create a covalent bond between the carbon atom C of the —C(Y)— group and said nucleophile;
wherein the ingredients i) and ii) are each packaged in a separate packaging assembly.
